(19) United States
(12) Patent Application Publication
(10) Pub. No.: US 2026/0327399 A1
(43) Pub. Date: Oct. 22, 2026

(54) METHOD FOR PRODUCING AQUEOUS POLYMER DISPERSIONS FROM ORGANIC WASTE MATERIALS

(71) Applicant: BASF SE, Ludwigshafen am Rhein (DE)

(72) Inventors: Wolfgang HUEBINGER, Ludwigshafen am Rhein (DE); Bernhard SCHULER, Ludwigshafen am Rhein (DE); Joachim PAKUSCH, Ludwigshafen am Rhein (DE); Konrad ROSCHMANN, Ludwigshafen am Rhein (DE); Stephan AMTHOR, Ludwigshafen am Rhein (DE); Eric NEUHAUS, Ludwigshafen am Rhein (DE); Daniel KEHRLOESSER, Ludwigshafen am Rhein (DE); Maximilian RUELLMANN, Ludwigshafen am Rhein (DE)

(21) Appl. No.: 19/110,447

(22) PCT Filed: Sep. 7, 2023
(86) PCT No.: PCT/EP2023/074589

§ 371 Date: Mar. 11, 2025

(30) Foreign Application Priority Data
Sep. 12, 2022 (EP) 22195134.6

(51) Int. Cl. C08J 11/12; C01B 3/34; C07C 29/151; C07C 31/04; C08J 11/14

(52) U.S. Cl. CPC C08J 11/12; C01B 3/34; C07C 29/151; C07C 31/04; C08J 11/14; C08J 2331/02; C08J 2333/08; C08J 2333/10

(57) ABSTRACT

The present invention relates to a process for producing aqueous polymer dispersions by radical aqueous emulsion polymerization of at least one ethylenically unsaturated monomer M, where a portion of the monomers M to be polymerized are ethylenically unsaturated monomers M′ produced from a pyrolysis oil as a raw material, which is obtained by pyrolysis of plastic waste materials, which are in particular selected from mixed plastic waste and waste rubber. In particular, the present invention relates to such a radical aqueous emulsion polymerization of monomers M which is carried out on industrial scale.

Method for producing aqueous polymer dispersions from organic waste materials.

The present invention relates to a process for producing aqueous polymer dispersions by radical aqueous emulsion polymerization of at least one ethylenically unsaturated monomer M, where at least a portion of the monomers M to be polymerized are ethylenically unsaturated monomers M′ produced from a raw material which is obtained by pyrolysis of organic waste materials.

Aqueous polymer dispersions of polymerized ethylenically unsaturated monomers, also referred to as polymer latices, are fluid systems comprising dispersed polymer particles of a chain growth addition polymer in an aqueous dispersing medium. Depending on the polymer architecture of the dispersed polymer particles, the polymer dispersions can be used across a plethora of technical applications, including binders for paints, architectural as well as industrial coatings, binders for paper coatings, binders in adhesives, binders for fiber bonding, organic opacifiers, rubbers and impact modifiers for thermoplastics.

Aqueous polymer dispersions are typically produced by aqueous emulsion polymerization, in particular prepared by a free radical aqueous emulsion polymerization of ethylenically unsaturated monomers, which include monovinyl aromatics such as styrene or vinyl toluene, mono- and diolefins, such as butadiene, isoprene or ethene, esters of α, β-ethylenically unsaturated acids, in particular the esters of acrylic acid or methacrylic acid, vinyl or allyl ethers and vinyl or allyl esters, and combinations thereof. The term “free radical aqueous emulsion polymerization” means that the polymerization of the monomers M is initiated by radicals formed by the decay of a polymerization initiator, whereby free radicals are formed in the polymerization mixture. It is therefore also termed “radically initiated emulsion polymerization”. The procedure for radically initiated emulsion polymerizations of monomers in an aqueous medium has been extensively described and is therefore sufficiently familiar to the skilled person [cf. in this regard Emulsion Polymerization in Encyclopedia of Polymer Science and Engineering, vol. 8, pages 659 ﬀ. (1987); D. C. Blackley, in High Polymer Latices, vol. 1, pages 35 ﬀ. (1966); H. Warson, The Applications of Synthetic Resin Emulsions, chapter 5, pages 246 ﬀ. (1972); D. Diederich, Chemie in unserer Zeit 24, pages 135 to 142(1990 ); Emulsion Polymerisation, Interscience Publishers, New York (1965); DE-A 40 03 422; and Dispersionen synthetischer Hochpolymerer, F. Hölscher, Springer-Verlag, Berlin (1969), D. Urban, K. Takamura (ed.) “Polymer Dispersions and Their Industrial Applications”, Wiley VCH, Weinheim 2002)]. Detailed mechanistic studies have been summarized e.g. by C. S. Chern, Prog. Polym. Sci. 31(2006 ) 443-486; and by M. Nomura et al., Adv. Polym. Sci. 175(2005 ) 1-128.

So far, the ethylenically unsaturated monomers used in the emulsion polymerization are based on fossil naphtha sources obtained from crude oil refinery. For the production of the monomers, fossil naphtha is first subjected to (hydro) cracking to obtain ethylene, propylene, C4 olefin cuts, including in particular 1-butene, isobutene and butadiene, higher olefin cuts, hereinafter also referred to as >C4 olefin cuts, typically C5-C10 olefins, and aromatic cuts, including in particular benzene, toluene, xylenes and ethylbenzene. The monomers used for the emulsion polymerization are obtained from the aforementioned products of the (hydro) cracking process optionally in combination with syngas (cf. K. Weissermel, H. -J. Arpe, Industrial Organic Chemistry, 4^{th} edition 1994, VCH Verlagsgesellschaft mbH Weinheim). Like the (hydro) cracking products, syngas is typically prepared from fossil sources, e.g. by steam reforming of methane obtained from gas fields or by gasification of coal.

Plastic waste is an increasing environmental problem. Currently, plastic waste is still largely landfilled or incinerated for heat generation. Chemical recycling is an attractive way to convert waste plastic material into useful chemicals. An important technique for chemically recycling plastic waste is pyrolysis. The pyrolysis is a thermal degradation of plastic waste in an inert atmosphere and yields value added products such as pyrolysis gas, liquid pyrolysis oil and char (residue), wherein pyrolysis oil is the major product. The pyrolysis oil can be used as source for syngas production and/or processed into chemical feedstock such as ethylene, propylene, C4 olefine cuts, >C4 olefine cuts and aromatic cuts etc. for example in a (steam) cracker.

The production of pyrolysis oil and its use as a feed stock in (steam) cracker has been described frequently, e.g. in WO 95/03375, EP 713906, WO 2015/128033, WO 2020/008050 and by Y. Zhang et al., Fuel Processing Technology 206(2020 ) 106455 (doi.org/10.1016/j.fuproc.2020.106455). WO 2021/224287 describes a process for purificaton of pyrolysis oil obtained by pyrolysis of plastic waste.

US 2021/0139620 describes a process for producing polystyrene, which comprises dehydrogenation of an alkylbenzene which is directly or indirectly obtained from a pyrolysis oil produced by pyrolyzing recycled waste such as waste plastic, to obtain styrene, which is then used in the production of the polystyrene.

WO 2022/016177 describes the conversion of plastics into monomers by pyrolysis. The process comprises heating a plastic feed stream to a temperature of 300 to 600 °C to pyrolize the waste plastic, subjecting a portion of the thus obtained low temperature product stream to a second pyrolysis at a temperature of 600 to 1100 °C to obtain a high temperature product stream and recovering the monomers from the high temperature product stream. However, only C2-C4 olefines and styrene can be obtained by this process.

Unfortunately, pyrolysis oil obtained from plastic waste contains contaminants which may cause problems in the downstream processing of the pyrolysis oil. This is because the polymers contained in plastic waste are typically composed not only of carbon and hydrogen but contain other elements such as halogens, sulfur, phosphor, nitrogen and oxygen stemming from the reactants used in the production of the polymers, i.e. monomers, initiators, polymerization catalysts, surfactants and chain transfer agents. Mixed waste plastic may, however, also contain additives, such as processing aids, stabilizers, dyes, pigments and flame retardants which also contribute to the amount of organic bound elements other than hydrogen and carbon in the pyrolysis oil. Waste plastic can also be contaminated in other ways, for example by food residues or by product residues in packaging, e.g. in the case of paint cans or building material containers. These contaminants will also contribute to the amount of organic bound elements other than hydrogen and carbon in the pyrolysis oil.

Although the pyrolysis oil is frequently purified before subjecting it to further processing, some impurities remain persistent. Organosulphur compounds are still found in fossil naphtha, despite multiple cleaning operations in refineries, although they are close to the detection limit. Pyrolysis oil contains significantly higher amounts of organosulfur and elemental sulfur than fossil naphtha, e.g. pyrolysis oil based on used tyres contains up to 1% sulphur compounds. Despite further purification operations also at the base product and at monomer stage, the monomers produced from pyrolysis oil will likely contain higher traces of sulphur, possibly below the detection limit and other trace impurities, compared to monomers produced from fossil naphtha. Moreover, pyrolysis oil may contain a considerable amount of organic bound oxygen (so called oxygenates), such as aldehydes, ketones, phenolic compounds, quinones and peroxides and nitrogen containing compounds, such as nitroso compounds.

Radical polymerisations are very sensitive to the smallest trace impurities. Emulsion polymerisations are even more sensitive than single-phase polymerisations due to interfacial effects. It is known that oxygenates can accelerate emulsion polymerisation (peroxides) or retard it (aldehydes, ketones). Phenolic compounds and quinones may even inhibit a radical polymerization. Due to the polarity of these molecules, they preferentially accumulate at interfaces and, once introduced, can remain quite persistently on e.g. the metallic surfaces of reactors (cf. W. Hübinger: Start behaviour of emulsion polymerisation, diploma thesis, TU Berlin 1985). Nitroso compounds may be co-stabilizers of inhibitors. Sulphur compounds are also surface-active and have a considerable influence on polymerisation. Even traces below 1 ppm can have a chain-regulating effect, cause colour changes or perceptibly alter the odour of the product. Like oxygenates they can remain quite persistently on e.g. the metallic surfaces of reactors.

If such trace impurities are present in the aqueous phase, they will affect the onset of polymerisation and thus alter particle formation. This may especially affect the properties of polymer dispersions, e.g. particle-size distribution or latex viscosity which may be problematic in terms of pigment/binder interactions and, thus coloristics for architectural coatings or in terms of thickener response for fast-running adhesive or paper-coatings dispersions, especially when batch-to-batch variations are large. Although some of the aformentioned compounds are deliberately used as chain regulators for tailoring the properties of the polymer dispersions, trace impurities are generally believed to cause troubles during emulsion polymerization such as flocculation or the formation of deposits which are a major problem in polymer dispersion production leading to high cleaning efforts and may cause speck formation. Apart from that, impurities are believed to increase the risk of an uncontrolled polymerisation and coagulation of the polymer dispersion, causing clogging of the reactor. These trace substances can often hardly be detected analytically but still can recognised by their influence on the starting behaviour of the polymerisation (see e.g. DE 4414082). Therefore, it is generally recommended for emulsion polymerization to keep the concentration of trace substances as low as possible in order to avoid troubles during emulsion polymerisation. Therefore, it must be assumed that pyrolysis oils obtained from plastic waste are not a suitable raw material base for monomers to be polymerised in an industrial-scale emulsion polymerisation process.

It was now surprisingly found that the use of ethylenically unsaturated monomers M, where at least a portion, in particular at least 20% by weight, preferably at least 30% by weight, more preferably at least 50% by weight, especially at least 50% by weight or up to 100% by weight of the monomers M to be polymerized in a radical aqueous emulsion polymerization are ethylenically unsaturated monomers M′ produced from a pyrolysis oil as a raw material, which is obtained by pyrolysis of plastic waste materials results in a reduced formation of deposits in their emulsion polymerization, in particular, if the radical aqueous emulsion polymerization is carried out on industrial scale.

Therefore, the present invention relates to a method for producing an aqueous polymer dispersion by radical aqueous emulsion polymerization of at least one ethylenically unsaturated monomer M, where a portion of the monomers M to be polymerized are ethylenically unsaturated monomers M′ produced from a pyrolysis oil as a raw material, which is obtained by pyrolysis of plastic waste materials, which are in particular selected from mixed plastic waste and waste rubber. In particular, the present invention relates to such a radical aqueous emulsion polymerization of monomers M which is carried out on industrial scale.

The present invention is associated with particular benefits. Since the use of ethylenically unsaturated monomers M′ obtained from pyrolysis oil of plastic waste in a radical aqueous emulsion polymerization result in reduced formation of deposits, product quality of the polymer dispersion may be improved and the time period between the purification cycles of reaction vessels used in the technical scale radical aqueous emulsion polymerization of ethylenically unsaturated monomers can be increased resulting in improved space/time yields of such products.

Therefore, the present invention also relates to the use of ethylenically unsaturated monomers M′ produced from a pyrolysis oil as a raw material, which is obtained by pyrolysis of plastic waste materials, which are in particular selected from mixed waste plastic and waste rubber, for increasing the time period between the purification cycles of reaction vessels used in the industrial scale radical aqueous emulsion polymerization of ethylenically unsaturated monomers.

Without being bound by theory, it is believed that the benefits achieved by monomers produced from pyrolysis oil feedstock result from trace impurities, such as organic sulfur compounds, oxygenates and organic nitrogen compounds, which stem from the pyrolysis oil feedstock, despite the numerous purification and reaction steps. As mentioned before, such trace impurities are principally capable of inhibiting or regulating a radical polymerization reaction und thus assumed in the past that such impurities should not be present in the substances used for emulsion polymerization if possible, in order to ensure a reaction system that is as defined as possible. It appears, however, that these impurities accumulate in the interface of the growing polymer particles and prevent an uncontrolled overreaction of the monomers there, thus reducing the formation of coagulate and thus the formation of deposits.

Hereinafter, the invention is explained in more detail.

In the context of the present invention, the term “plastic waste” refers to any plastic material discarded after use, i.e., the plastic material has reached the end of its useful life. The plastic waste can be pure polymeric plastic waste, mixed plastic waste (also referred to as mixed waste plastic) or film waste, including soiling, adhesive materials, fillers, residues etc. The plastic waste has a nitrogen content, sulfur content, halogen content and optionally also a heavy metal content. The plastic waste can originate from any plastic material containing source. Accordingly, the term “plastic waste” includes industrial and domestic plastic waste including used tires and agricultural and horticultural plastic material. The term “plastic waste” also includes used petroleum-based hydrocarbon material such as used motor oil, machine oil, greases, waxes, etc. In the context of the present invention, the “plastic waste” to be pyrolyzed preferably is mixed plastic waste. The term plastic waste also includes waste rubber, in particular waste rubber resulting from tires. The term plastic waste also includes pure polymeric plastic waste, or film waste, including soiling, adhesive materials, fillers, residues, etc. IN particular, plastic waste includes mixed plastic waste and waste rubber, in particular waste rubber from tires.

Typically, plastic waste is a mixture of different plastic material, including hydrocarbon plastics, e.g., polyolefins such as polyethylene (HDPE, LDPE) and polypropylene, polystyrene and copolymers thereof, etc., and polymers composed of carbon, hydrogen and other elements such as chlorine, fluorine, oxygen, nitrogen, sulfur, silicone, etc., for example chlorinated plastics, such as polyvinylchloride (PVC), polyvinylidene chloride (PVDC), etc., nitrogen-containing plastics, such as polyamides (PA), polyurethanes (PU), acrylonitrile butadiene styrene (ABS), etc., oxygen-containing plastics such as polyesters, e.g., polyethylene terephthalate (PET), polycarbonate (PC), etc.), silicones and/or sulfur bridges crosslinked rubbers. PET plastic waste is often sorted out before pyrolysis, since PET has a profitable resale value. Accordingly, the plastic waste to be pyrolyzed often contains less than about 10 wt.-%, preferably less than about 5% by weight and most preferably substantially no PET based on the dry weight of the plastic material. One of the major components of waste from electric and electronic equipment are polychlorinated biphenyls (PCB). Typically, the plastic material comprises additives, such as processing aids, plasticizers, flame retardants, pigments, light stabilizers, lubricants, impact modifiers, antistatic agents, antioxidants, etc. These additives may comprise elements other than carbon and hydrogen. For example, bromine is mainly found in connection to flame retardants. Heavy metal compounds may be used as lightfast pigments and/or stabilizers in plastics; cadmium, zinc and lead may be present in heat stabilizers and slip agents used in plastics manufacturing. The plastic waste can also contain residues. Residues in the sense of the invention are contaminants adhering to the plastic waste. The additives and residues are usually present in an amount of less than 50 wt.-%, preferably less than 30 wt.-%, more preferably less than 20 wt.-%, even more preferably less than 10% by weight, based on the total weight of the dry weight plastic.

In the context of the present invention, the term “pyrolysis” relates to a thermal decomposition or degradation of end-of-life plastics under inert conditions and results in a gas, a liquid and a solid char fraction. During the pyrolysis, the plastics are converted into a great variety of chemicals including gases such as H₂, C₁-C₄-alkanes, C₂-C₄-alkenes, ethyne, propyne, 1-butyne, pyrolysis oil having a boiling temperature of 25 °C to 500 °C and char. The term “pyrolysis” includes slow pyrolysis, fast pyrolysis, flash catalysis and catalytic pyrolysis. These pyrolysis types differ regarding process temperature, heating rate, residence time, feed particle size, etc. resulting in different product quality.

In the context of the present invention, the abbreviated notation (steam) cracking includes both thermal cracking such as steam cracking and catalytic cracking such as catalytic hydrocracking and fluidized catalytic cracking (FCC). In a similar manner, the abbreviated notation (steam) cracker includes a thermal cracking reactor such as steam cracker, and a catalytic cracking reactor, such as a catalytic hydrocracking reactor and a fluidized catalytic cracking reactor.

In the context of the invention, the term “sulfur content” relates to the content of sulfur, selected from H₂S, elemental sulfur and organic sulfur, determined according to ASTM D 7183-18 and calculated as elemental sulfur.

In the context of the invention, the term “nitrogen content” relates to the content of organic nitrogen, determined according to ASTM D 5762-18 and calculated as elemental nitrogen.

In the context of the invention, the term “oxygen content” relates to the content of organic oxygen, determined according to ASTM D 7423-17 and calculated as elemental oxygen.

In the context of the present invention, the term “ppm” means 1 part per 1 million parts on a weight basis, i.e. 1 ppm=1 mg/kg.

In the context of the present invention, the term “pphm” means parts per 100 parts of monomers and refers to the relative amount of a monomer with respect to the total weight of monomers to be polymerized. 1 pphm of a monomer corresponds to 1% by weight of the monomer, based on the total weight of monomers to be polymerized.

In the context of the present invention, the term “industrial scale” is used synonymously to the terms “technical scale” and “large scale”. In the context of the radical aqueous emulsion polymerization “industrial scale” is understood that the emulsion polymerization is carried out in reactors having an internal volume of at least 0.1m³ , in particular at least 0.5m³ , e.g. in the range of 6 to 200m³ . A radical aqueous emulsion polymerization “industrial scale” can be carried out batch-wise, including monomer feed batch processes, and continuously.

Suitable crude pyrolysis oil originates from the pyrolysis of plastic waste. The plastics material used as feedstock for the production of said pyrolysis oil can be derived from any source comprising end of life plastic material. The content of sulfur, nitrogen, halogen and, if present, heavy metal of the pyrolysis oil can vary and depends on the type of the waste plastic material processed and pyrolysis conditions employed. For example, mixed waste plastic may result in a pyrolysis oil having a comparatively low sulfur content, e.g. in the range of 5 to 400 ppm, in particular 50 to 300 ppm, while pyrolysis oil obtained from wased rubber may have a significantly higher sulfur content, which may range from 400 to 50000 ppm, in particular 500 to 12000 ppm, on a weight basis.

The pyrolysis oil is produced by conventional pyrolysis processes known in the art. Pyrolysis processes for waste plastic as such are known. They are described, e.g. in WO 95/03375, EP 713906, WO 2015/128033 and WO 2020/008050. Suitable pyrolysis oils are also commercially available. Usually, the pyrolysis includes drying of the plastic waste at elevated temperatures but below 400 °C, e.g. in the range of 150 to 380 °C followed by pyrolysis at temperatures above 400 °C, e.g. in the range of 450 to 800 °C.

The crude pyrolysis oil obtained from the pyrolysis of plastic waste typically is a liquid at 15 °C. “Liquid at 15 °C” in the terms of the present invention means that the pyrolysis oil has a kinematic viscosity at 20 °C of at most 1000mm² /s at 20 °C, e.g. in the range of 20 to 1000 mm² /s at 20 °C. The pyrolysis oil typically has a density of at most of 1.3 g/mL, e.g. a density in the range from 0.7 to 1.1 g/mL, at 15 °C and 1013 mbar, as determined according to DIN EN ISO 12185. Frequently, the crude pyrolysis oil obtained from the pyrolysis of plastic waste has a boiling temperature of more than 35 °C and frequently of at most 500 °C. Typically the pyrolysis oil has a broad boiling range. Typically less than 10% by weight of the pyrolysis oil boil at temperatures below 100 °C and less than 10% by weight of the pyrolysis oil boil at temperature of 360 °C or higher at atmospheric pressure (1 bar). In particular at least 80% by weight of the pyrolysis oil boil in the range of 100 to 360 °C at atmospheric pressure (1 bar). The oil phase of the crude pyrolysis oil typically contains less than 100 ppm of dissolved water. Its pks value (at 20 °C) is frequently in the range of 4 to 8.

Depending on the waste plastic material subjected to the pyrolysis, the crude pyrolysis oil may have varying contents of sulfur, e.g. in the form of H₂S, elemental sulfur and/or organic sulfur, organic nitrogen, organic oxygen, halogen and, if present, heavy metal.

In the context of the present invention, the term “heavy metal” refers to a metal or metalloid having a density >4.51 g/cm³ (at 20 °C). Examples include arsenic, antimony, bismuth, selenium, tin, cadmium, chromium, iron, copper, mercury, nickel and lead.

The crude pyrolysis oil has typically at least one or all of the following properties:
(α) a content of sulfur, selected from H₂S, elemental sulfur and organic sulfur, of at least 30 ppm, in particular at least 40 ppm, e.g. in the range of 30 to 20000 ppm, frequently in the range of 40 to 15000 ppm, or in the range of 40 to 12000 ppm, determined according to ASTM D 7183-18 and calculated as elemental sulfur;
(β) content of organic nitrogen of at least 20 ppm, in particular at least 30 ppm, e.g. in the range of 20 to 40000 ppm, frequently in the range of 30 to 20000 ppm, or in the range of 40 to 10000 ppm, determined according to ASTM D 5762-18 and calculated as elemental nitrogen; and/or
(χ) a content of organic oxygen of at least 50 ppm, in particular at least 70 ppm or at least 80 ppm, e.g. in the range of 50 to 5000 ppm, frequently in the range of 70 to 4500 ppm or in the range of 80 to 4000 ppm, determined according to ASTM D 7423-17 and calculated as elemental oxygen.

In one embodiment, the crude pyrolysis oil has a halogen content of 40 mg/l or more, such as 80 ppm or more; or 120 ppm or more; or 400 ppm or more; or 600 ppm or more, relative to the total weight of the crude pyrolysis oil. In another embodiment, the crude pyrolysis oil has a halogen content of 100 to 1000 ppm, often 120 to 900 ppm, relative to the total weight of the crude pyrolysis oil.

Organofluorine, organochlorine, organobromine and/or organoiodine compounds typically are the source for the halogen content in the crude pyrolysis oil. Specifically, the halogen content is a bromine and chlorine content to 90% or more, such as 95% or more or even 100%. More specifically, the halogen content is to 90% or more, such as 95% or more or even 100% a chlorine content.

In case that the crude pyrolysis oil also has a heavy metal content, the heavy metal content is at least 1 ppm, relative to the total weight of the crude pyrolysis oil.

Frequently, the heavy metal content does not exceed 100 ppm, relative to the total weight of the crude pyrolysis oil. In one embodiment, the crude pyrolysis oil has a content of heavy metals other than iron in the range of 1 ppm to 4 ppm, or 1 to 3 ppm relative to the total volume of the crude pyrolysis oil. An iron content of 10 ppm or even more is often unavoidable due to the waste processing, e.g. residues from shredder-process and abrasion of steel apparatus like mills. Iron salts are often used in initiation of the polymerization, their dosage in the subsequent radical emulsion polymerisatioin can be adjusted.

The acid number of the crude pyrolysis oil obtained from plastic waste is typically in the range of 1 to 50 mg KOH/g, in particular in the range of 2 to 40 mg KOH/g, as determined by potentiometric titration in accordance with DIN EN 12634: 1999 but using tetrabutyl ammonium hydroxide instead of tetramethyl ammonium hydroxide.

In a preferred embodiment, the crude pyrolysis oil is obtained from mixed waste plastic. In this case, the crude pyrolysis oil has typically at least one or all of the following properties:
(α.1) a content of sulfur, selected from H₂S, elemental sulfur and organic sulfur, of at least 20 ppm, in particular at least 30 ppm, e.g. in the range of 20 to 5000 ppm, frequently in the range of 30 to 3000 ppm, determined according to ASTM D 7183-18 and calculated as elemental sulfur;
(β.1) a content of organic nitrogen of at least 20 ppm, in particular at least 30 ppm, e.g. in the range of 20 to 40000 ppm, frequently in the range of 30 to 10000 ppm, determined according to ASTM D 5762-18 and calculated as elemental nitrogen; and/or
(χ.1) a content of organic oxygen of at least 50 ppm, in particular at least 70 ppm or at least 80 ppm, e.g. in the range of 50 to 5000 ppm, frequently in the range of 70 to 4500 ppm or in the range of 80 to 4000 ppm, determined according to ASTM D 7423-17 and calculated as elemental oxygen.

The halogen content for a crude pyrolysis oil obtained from mixed waste plastic is usually as described above. Likewise, the acid number for a crude pyrolysis oil obtained from mixed waste plastic is usually as described above.

In another preferred embodiment, the crude pyrolysis oil is obtained from waste rubber. In this case, the crude pyrolysis oil has typically at least one or all of the following properties:
(α.2) a content of sulfur, selected from H₂S, elemental sulfur and organic sulfur, of at least 300 ppm, in particular at least 400 ppm, e.g. in the range of 300 to 20000 ppm, frequently in the range of 400 to 15000 ppm, determined according to ASTM D 7183-18 and calculated as elemental sulfur;
(β.2) a content of organic nitrogen of at least 300 ppm, in particular at least 350 ppm, e.g. in the range of 300 to 40000 ppm, frequently in the range of 350 to 20000 ppm, determined according to ASTM D 5762-18 and calculated as elemental nitrogen; and/or
(χ.2) a content of organic oxygen of at least 50 ppm, in particular at least 70 ppm or at least 80 ppm, e.g. in the range of 50 to 5000 ppm, frequently in the range of 70 to 4500 ppm or in the range of 80 to 4000 ppm, determined according to ASTM D 7423-17 and calculated as elemental oxygen.

The halogen content for a crude pyrolysis oil obtained from waste rubber is usually as described above. Likewise, the acid number for a crude pyrolysis oil obtained from waste rubber is usually as described above.

The crude pyrolysis oil may be purified before it is further processed, e.g. by stripping, one or more distillation steps, by contacting with trapping agents or by combined measures. A particular process for purification is described in WO 2021/224287, to which reference is made, and which is in particular applied for purification of the crude pyrolysis oil obtained from plastic waste.

The pyrolysis oil obtained from the purification usually has a sulfur content at least 40% lower, more preferably at least 50% lower, than that of the untreated crude pyrolysis oil.

The pyrolysis oil obtained from the purification usually has a nitrogen content at least 40% lower, more preferably at least 50% lower, than that of the untreated crude pyrolysis oil.

The pyrolysis oil obtained from the purification usually has an oxygen content at least 40% lower, more preferably at least 50% lower, than that of the untreated crude pyrolysis oil.

The pyrolysis oil obtained from the purification usually has a halogen content at least 40% lower, more preferably at least 50% lower, than that of the untreated crude pyrolysis oil.

If present, the pyrolysis oil obtained from the purification usually has a heavy metal content at least 40% lower, more preferably at least 50% lower, than that of the untreated crude pyrolysis oil.

For producing the monomers M′ used in the emulsion polymerization of the process of the present invention, the pyrolysis oil obtained from pyrolysis of plastic waste is further processed, e.g. by (steam) cracking or by gasification. For this, the gas fraction formed in the pyrolysis may also be used as a co-feed. The solid fraction obtained in the pyrolysis may also be used as a feedstock for the gasification to syngas.

The pyrolysis oil obtained from mixed waste plastic material is particularly suitable as a feedstock for (steam) cracker. Therefore, a preferred embodiment of the invention relates to a process, where the pyrolysis oil is obtained from mixed waste plastic material is subjected to (steam) cracking.

As mentioned above, (steam) cracking includes both thermal steam cracking and catalytic cracking. They are known methods for producing lower molecular weight hydrocarbon compounds from naphtha feedstock [cf. K. Weissermel, H. -J. Arpe, loc. cit., chapter 3, pp 65-98 and references cited therein) and can be applied by analogy to the (steam) cracking of pyrolysis oil obtained from mixed waste plastic material.

As mentioned above, (steam) cracking produces C₂-C₄ olefin fractions, such as ethylene, propene, C₄ olefin fractions including 1-butene, 2-butene, isobutene and butadiene, >C₄ olefin fractions, in particular C₅-C₁₀ monoolefins and aromatic fractions, including benzene and/or methyl benzene fractions. These products, optionally in combinations with syngas are the starting materials for most of the ethylenically unsaturated monomers M polymerized in a radical aqueous emulsion polymerization.

The pyrolysis oil obtained from the pyrolysis of mixed waste plastic material may be used as a feedstock as such or may be used in combination with other hydrocarbon feedstock, e.g. naphtha from fossil sources. To achieve the beneficial effects, the amount of pyrolysis oil obtained from the pyrolysis of mixed waste plastic material is at least 10%, in particular at least 20%, based on the total amount of feedstock subjected to (hydro) cracking.

The feedstock which is subjected to (steam) cracking is typically a full range naphtha obtained from pyrolysis oil or a mixture of full range naphtha obtained from pyrolysis oil and conventional naphtha feedstock. In the context of the present invention, the term “full range naphtha refers to the fraction of pyrolysis oil or fossil oil which has a has a boiling temperature of in the range of 35 to 210 °C, in particular in the range of 50 to 200 °C at atmospheric pressure (1 bar).

In particular, the feedstock subjected to (steam) cracking meets at least one or all of the following requirements:
(α.3) a content of sulfur, selected from H₂S, elemental sulfur and organic sulfur, of at least 5 ppm, in particular at least 10 ppm, e.g. in the range of 5 to 500 ppm, frequently in the range of 10 to 400 ppm, determined according to ASTM D 7183-18 and calculated as elemental sulfur;
(β.3) a content of organic nitrogen of at least 20 ppm, in particular at least 30 ppm, e.g. in the range of 20 to 10000 ppm, frequently in the range of 30 to 4000 ppm, determined according to ASTM D 5762-18 and calculated as elemental nitrogen; and/or
(χ.3) a content of organic oxygen of at least 30 ppm, in particular at least 50 ppm or at least 60 ppm, e.g. in the range of 30 to 3000 ppm, frequently in the range of 50 to 2000 ppm or in the range of 60 to 1500 ppm, determined according to ASTM D 7423-17 and calculated as elemental oxygen.

The feedstock which is subjected to (steam) cracking usually has a halogen content at most 20 ppm of halogen.

The feedstock which is subjected to (steam) cracking usually has content of heavy metals other than iron of less than 1 ppm.

Frequently, the feedstock which is subjected to (steam) cracking has a water content of not more than 100 ppm. Its pKs value (at 20 °C) is typically in the range of 4 to 8.

The pyrolysis oil obtained from the pyrolysis of plastic waste, in particular of waste rubber such as tire or construction rubber may also be used as a feedstock for the production of syngas. Therefore, a preferred embodiment of the invention relates to a process, where the pyrolysis oil is obtained from plastic waste, in particular from waste rubber material is subjected to gasification in a syngas process. In the syngas process, a mixture of carbon monoxide and hydrogen, which is then used in the production of the ethylenically unsaturated monomers, optionally in the combination with fractions of the (steam) cracking.

In the context of the present invention, syngas, which is also known as synthesis gas, is a mixture of gases comprising as main components carbon monoxide (CO) and hydrogen (H₂). The preparation of syngas has been known for long time [cf. K. Weissermel, H. -J. Arpe, loc. cit., chapter 2.1 and 2.2, pp 15-30 and references cited therein). For the production of syngas, the pyrolysis oil obtained from pyrolysis of plastic waste, in particular from the pyrolysis of waste rubber, is partially oxidized with oxygen (O₂), air, steam (H₂O), or a combination of all gasification agents at elevated temperature, often in the range from 800 to 1000 °C and at a pressure in the range from 1 to 20 bar. The obtained syngas is then used in the production of ethylenically unsaturated monomers. In particular, the syngas is then used in the production of methanol which is subsequently used in the production of the ethylenically unsaturated monomers M′, e.g. by the so-called MTO route (methanol to olefins). A portion of that syngas-based methanol may also be converted into alkanes, olefins, oxygenates, and alcohols. These chemicals can be blended into, or used directly as, diesel fuel, gasoline, and other liquid fuels.

The pyrolysis oil obtained from the pyrolysis of plastic waste, in particular of waste rubber may be used as a feedstock as such or may be used in combination with other organic feedstock for syngas production, e.g. HVR (high vacuum residue) from fossil sources. To achieve the beneficial effects, the amount of pyrolysis oil obtained from the pyrolysis of plastic waste, in particular of waste rubber material is at least 10%, in particular at least 20%, based on the total amount of feedstock subjected to syngas production.

The feedstock which is subjected to syngas production, i.e. the pyrolysis oil or the mixture of pyrolysis oil and other organic feedstock, preferably meets at least one or all of the following requirements:
(α.4) a content of sulfur, selected from H₂S, elemental sulfur and organic sulfur, of at least 300 ppm, in particular at least 400 ppm, e.g. in the range of 300 to 15000 ppm, frequently in the range of 400 to 10000 ppm, determined according to ASTM D 7183-18 and calculated as elemental sulfur;
(β.4) a content of organic nitrogen of at least 300 ppm, in particular at least 350 ppm, e.g. in the range of 300 to 40000 ppm, frequently in the range of 350 to 20000 ppm, determined according to ASTM D 5762-18 and calculated as elemental nitrogen; and/or
(χ.4) a content of organic oxygen of at least 50 ppm, in particular at least 70 ppm or at least 80 ppm, e.g. in the range of 50 to 5000 ppm, frequently in the range of 70 to 4500 ppm or in the range of 80 to 4000 ppm, determined according to ASTM D 7423-17 and calculated as elemental oxygen.

The feedstock which is subjected to syngas production usually has a halogen content at most 20 ppm of halogen.

The feedstock which is subjected to (steam) cracking usually has heavy metal content of less than 1 ppm.

Usually, the feedstock which is subjected to gasification has a higher boiling range than the feedstock used for (steam) cracking. In particular, it has a boiling temperature of 200 °C or more, preferably 350 °C or more, at atmospheric pressure. Suitable feedstocks are the pyrolysis oil obtained from plastic waste, in particular from waste rubber, or a mixture of pyrolysis oil obtained from plastic waste, in particular from waste rubber, with HVR. Generally, the feedstock for the production of syngas has a water content of not more than 100 ppm. Its pKs value (at 20 °C) is typically in the range of 4 to 8.

Apparently, the pyrolysis oil obtained from the pyrolysis of plastic waste is directly usable as a feedstock for gasification to provide CO and H₂.

For (steam) cracking, the pyrolysis oil obtained from the pyrolysis of plastic waste requires reduction of the upper boiling point and cleaning to get a feedstock, which provides the starting materials for the production of monomers:
C₂-C₄-olefine fraction, in particular ethylene, propylene, 1-butene, 2-butene, isobutene, and butadiene;
an aromatic fraction, in particular, benzene, toluene, ethylbenzene, and xylenes;
a >C₄ olefin fraction, in particular a C₅-C₁₀ olefins;
a methane fraction, which can also be used to produce syngas;

These fractions can be used as monomers as such in case of the C₂-C₄-olefine fraction, in particular in case of butadiene and ethylene or they can be converted to the desired monomers M′ for the production of ethylenically unsaturated monomers.

In particular, the pyrolysis of waste plastic allows for producing the aqueous polymer dispersions by the following sequences of process steps:
i) Providing a pyrolysis oil from organic waste materials selected from mixed waste plastic and waste rubber;
ii) providing a full range naphtha from the pyrolysis oil obtained in step i), which particularly meets the aforementioned properties, e.g. by fractionating the pyrolysis oil to obtain a feedstock having a boiling range at atmospheric pressure in the range of 35 to 210 °C, in particular in the range of 50 to 200 °C, and by cleaning the pyrolysis oil and/or said fraction;
iii) Subjecting this full range naphtha to a steam cracking where C₂-C₄ olefin fractions, >C₄ olefin fractions and benzene and/or methyl benzene fractions are obtained, optionally
iv) Converting at least one of the fractions obtained in step iii) into an ethylenically unsaturated monomer M′; and/or
ii.a) Subjecting the pyrolysis oil directly, i.e. without cleaning, to a gasification step to obtain a syngas comprising a mixture of carbon monoxide and hydrogen,
iii.a) Using the syngas fraction obtained in step ii.a) in the production of an ethylenically unsaturated monomer M′; and
v) conducting a radical aqueous emulsion polymerization of at least one ethylenically unsaturated monomer M, where preferably at least 20% by weight, more preferably at least 30% by weight and especially at least 50% by weight of the monomers M to be polymerized are selected from the monomers M′ obtained in step iv), or step iii.a) and one or more of the C₂-C₄ olefins obtained in step iii).

According to the invention, the fractions obtained in steps iii) and ii.a) respectively, can be combined with the gaseous fractions obtained in the pyrolysis of plastic waste, namely the C₂-C₄-olefines, acetylene, propyne and/or 1-butyne, and used as a feedstock for producing the monomers M′.

In particular, the following fractions and combinations of fractions (a)-(e) obtained from steam cracking or gasification are used for the production of ethylenically unsaturated monomers:
(a) the combination of the C₂-C₄-olefine fraction and the benzene fraction or methylbenzene fraction, which is in particular used for the production of monovinyl aromatic monomers, such as styrene, vinyl toluene and divinyl benzene and further cyclohexanol, where the latter can be used in the production of cyclohexyl esters of acrylic acid or methacrylic acid;
(b) a C₂-C₄-olefine fraction which is in particular used for the production of acrylic acid, acrylonitrile, acrylamide, vinyl propionate (via propionic acid) and for the production of higher olefins and >C₄ alkanols, e.g. C₅-C₁₀ alkanols, where the latter are used in the production of C₅-C₁₀ alkylesters of acrylic acid and C₅-C₁₀ alkylesters of methacrylic acid;
(c) the syngas fraction, which is in particular used in the production of methanol, which itself is used in the production of methyl methacrylate;
(d) a C₂-C₄-olefine fraction and the syngas fraction, which is in particular used for the production of methacrylic acid, methyl methacrylate, vinyl acetate, vinyl propionate and vinyl esters of Koch acids;
(e) a >C₄ olefin fraction which is in particular used for the production of >C₄ alcohols, such as C₅-C₁₀ alkanols and C₅-C₁₀ cycloalkanols, which are required for the production of the C₅-C₁₀ alkylesters of acrylic acid and C₅-C₁₀ alkylesters of methacrylic acid;
(f) a C₂-C₄-olefine fraction, a >C₄ olefin fraction and the syngas fraction which is used for the production of C₅-C₁₀ alkylesters of acrylic acid and C₅-C₁₀ alkylesters of methacrylic acid.

Apparently, the following ethylenically unsaturated monomers are readily available from pyrolysis oil obtained by the pyrolysis of plastic waste: monovinyl aromatic monomers, divinyl aromatic monomers, butadiene, acrylate esters, methacrylate esters, acrylic acid, methacrylic acid, amides of acrylic acid, amides of methacrylic acid, acrylonitrile, vinylesters of C₂-C₁₂-alkanoic acids and mixtures thereof.

Here and in the following, the acrylate ester are in particular selected from C₁-C₁₈ alkyl acrylates and C₅-C₁₈ cycloalkyl acrylates and where the methacrylate esters are selected from C₁-C₁₈ alkyl methacrylates and C₅-C₁₈ cycloalkyl methacrylates.

The production of ethylenically unsaturated monomers and building blocks for the production of the monomers M′ is further illustrated by way of non-limiting examples. Further monomers will be obtainable by analogous processes, e.g. hydroxyalkyl esters of ethylenically unsaturated carboxylic acids, such as the hydroxyalkyl esters of acrylic acid and the hydroxyalkyl esters of methacrylic acid.

### Styrene

Styrene is conventionally produced from ethylbenzene by vapor phase catalytic dehydrogenation in the presence of steam. The catalyst used for this reaction typically consists primarily of ferric oxide. Reaction temperatures are generally in the range of 550 to 630 °C. The resulting crude product is distilled to remove unreacted ethylbenzene and byproducts, such as toluene and benzene. The ethylbenzene used as starting product can in turn be prepared by Friedel-Crafts alkylation of benzene with ethylene in the presence of aluminum chloride or zeolite as catalyst. According to the invention, benzene and ethylene may be obtained by cracking the pyrolysis oil, as described herein.

An alternative commercial procedure for producing styrene is the so-called POSM (propylene oxide/styrene monomer) process. In the first step of this process ethylbenzene is peroxidized by treating with air at a temperature of usually about 145 °C. The resulting 1-hydroperoxyethylbenzene serves in the following step as an epoxidation reagent for converting propene to propylene oxide, and is itself transformed to 1-phenylethanol. This epoxidation reaction generally proceeds at a temperature of about 100 to 130 °C in the presence of a suitable catalyst, such as e.g. a soluble molybdenum salt. In the final step the obtained 1-phenylethanol is subjected to dehydration which proceeds at a temperature of typically about 200 °C in the presence of an acidic catalyst, e.g. alumina. The propene used in this procedure may be obtained as C₃-olefine fraction during cracking of the pyrolysis oil according to the invention.

A further alternative process for producing styrene is based on a catalytic side-chain alkylation of toluene with methanol that avoids the need of a dehydrogenation step. In comparison to the conventional process, this one requires less expensive starting materials and is more energy-efficient, but so far suffers from its low selectivity. Toluene may be obtained from the pyrolysis oil according to the invention.

### Divinylbenzene

Divinylbenzene, which is usually understood as the mixture of especially its meta- and para-isomers, can produced from diethylbenzene by vapor phase catalytic dehydrogenation in analogy to the procedure for producing styrene described above. Diethylbenzene, or a mixture of its positional isomers, is in turn accessible by ethylating ethylbenzene, benzene or a mixture thereof by means of a Friedel-Crafts alkylation analogous to the ethylation of benzene described before in connection with the production of styrene. In fact, diethylbenzene is an inadvertent side product of this process anyway. The starting materials benzene and ethylene may be obtained from the pyrolysis oil according to the invention.

### Acrylic Acid

At present, a two-step propene oxidation process is predominantly used for the large-scale production of acrylic acid. Both oxidation steps are highly exothermic and use air as oxidizing agent. In the first step, propene is oxidized typically in the presence of a bismuth-molybdenum oxide catalyst at temperatures of often about 350 °C to give propenal (acrolein). In the second step, propenal is oxidized to acrylic acid usually over a molybdenum-vanadium oxide catalyst at temperatures of often around 260 °C. The propene used in this process may be obtained from the pyrolysis oil according to the invention.

Another process for producing acrylic acid starts from carbon monoxide and ethylene oxide, the latter being accessible on an industrial scale via catalytic oxidation of ethylene. In a first step, ethylene oxide is catalytically carbonylated with carbon monoxide to yield propiolactone, which is thermolytically converted to acrylic acid in the presence of a suitable catalyst. Alternatively, propiolactone is first catalytically polymerized into polypropiolactone which is then degraded by pyrolysis to acrylic acid. The ethylene and the carbon monoxide used in this process may be obtained from the pyrolysis oil or the syngas according to the invention.

### Methacrylic Acid

In analogy to the aforementioned two-step propene oxidation process for the production of acrylic acid, methacrylic acid can be produced from isobutene in two catalytic oxidation steps. Accordingly, in the first oxidation step isobutene is converted to methacrolein, which is then oxidized to methacrylic acid. The catalysts used in both steps are usually similar to the corresponding ones used in the process for oxidizing propene to acrylic acid. The isobutene used in this procedure may be obtained from the pyrolysis oil according to the invention.

Methacrolein obtained in the first step of the above process can alternatively be produced from propionaldehyde, which is accessible by hydroformylation of ethylene with syngas the presence of a catalyst such as cobalt tetracarbonyl hydride or a rhodium complex. The propionaldehyde obtained is reacted with formaldehyde and catalytic amounts of dimethylamine to give the corresponding Mannich adduct, which then undergoes thermal release of the amine to yield methacrolein. As starting materials for this process, ethylene obtained from the pyrolysis oil according to the invention and the syngas according to the invention may be used.

### Methyl Methacrylate

A fairly new process starts from ethylene which is converted to methyl propionate via a homogeneously catalyzed carboxymethylation with carbon monoxide and methanol. In the next step, the methyl propionate is converted into methyl methacrylate by aldol condensation with formaldehyde in the gas phase using a supported catalyst, such as cesium oxide on silica. The ethylene and the carbon monoxide used in this process may be obtained from the pyrolysis oil or the syngas according to the invention, while methanol may be prepared as described herein below.

In an even newer process, which is still in the pilot phase, first methacrolein is produced using one of the processes described above in connection with the production of methacrylic acid. In a subsequent step, the methacrolein is simultaneously oxidized and esterified with atmospheric oxygen and methanol over a nickel-gold catalyst at moderate reaction conditions, such as typically a temperature of about 90 °C and a pressure of about 6 bar, to give methyl methacrylate. The methanol used in this process can be obtained as described herein below.

### Methanol

The technical production of methanol is performed by catalytic hydrogenation of syngas, which preferably has a ratio of carbon monoxide to hydrogen of about 1:2. The process is carried out at different pressures and temperatures depending on the type of catalyst used. In the so-called low-pressure process, which is mainly used at present, hydrogenation usually takes place at pressures of about 50 to 100 bar and temperatures of about 200 to 300 °C in the presence of a mixture of copper and zinc oxides supported on alumina as catalyst. The syngas according to the invention may be used as starting material of this process.

Methanol may be used for the production of methyl acrylate by the usually acid-catalyzed esterification with acrylic acid. Methanol can be also used via a methanol to olefin (MTO)-route to produce olefins, preferably ethylene and propylene (for a review on MTO see e.g. M. R. Gogate (2019) Methanol-to-olefins process technology: current status and future prospects, Petroleum Science and Technology, 37: 5, 559-565, DOI: 10.1080/10916466.2018.1555589).

### n-Propanol

n-Propanol is produced on a large scale by catalytic hydrogenation of propionaldehyde usually at temperatures of around 110-150 °C and pressures of about 1.5-10 bar in the presence of typically a supported copper-nickel-zinc-chromium catalyst. Propionaldehyde in turn is accessible by hydroformylation of ethylene as described above.

n-Propanol is useful for the production of n-propyl acrylate and n-propyl methacrylate either by a usually acid-catalyzed esterification of acrylic or methacrylic acid, or by transesterification with methyl acrylate or methyl methacrylate, respectively.

### n-Butanol and Isobutanol

On an industrial scale n-butanol and isobutanol are mainly produced by the following two-step process. The first step is a hydroformylation of propene with carbon monoxide and hydrogen in the presence a homogeneous cobalt or rhodium catalyst. In the second step the resulting mixture of n-butanal and 2-methylpropanal is hydrogenated to the corresponding mixture of n-butanol and isobutanol, which is then separated by distillation into the two individual alcohols. Since the demand for n-butanol is generally higher, especially the catalyst used in the hydroformulation is chosen so that the formation of n-butanal is favored over that of 2-methylpropanal. The starting compound propene may be obtained from the pyrolysis oil of the invention, while the syngas according to the invention may serve as the mixture of carbon monoxide and hydrogen used in the process.

n-Butanol and isobutanol are useful for the production of the corresponding n-butyl or isobutyl (meth)acrylates either by a usually acid-catalyzed esterification of acrylic or methacrylic acid, or by transesterification with methyl acrylate or methyl methacrylate, respectively.

### Tert-Butanol

The large-scale production of tert-butanol is carried out by acid-catalyzed hydration of isobutene typically at temperatures of about 30-120 °C and pressures of about 5-12 bar. Acidic ion exchange resins are mainly used as catalysts. The starting compound isobutene may be obtained from the pyrolysis oil according to the invention.

### 2-Ethylhexanol

The usual starting material for the industrial production of racemic 2-ethylhexanol is n-butanal, which is generated by hydroformylation of propene, as described above. The n-butanal is subjected to a self-aldol condensation to give 2-ethyl-hex-2-enal, which is then catalytically hydrogenated to 2-ethylhexanol.

2-Ethylhexanol is useful for the production of 2-ethylhexyl acrylate and 2-ethylhexyl methacrylate either by a usually acid-catalyzed esterification of acrylic or methacrylic acid, or by transesterification with methyl acrylate or methyl methacrylate, respectively.

### Cyclopentanol

Cylcopentanol can be produced by a 3-step procedure starting from 1,3-butadiene. In the first step the 1,3-butadiene is converted to adipic acid or an ester thereof via dicarbonylation with carbon monoxide in the presence of usually a homogeneous cobalt or palladium complex. Cyclopentanone can then be obtained by ketonization of adipic acid with a strong base, such as e.g. barium hydroxide, or by Dieckmann condensation of an adipic acid ester and subsequent decarboxylation. In the final step cyclopetanone is reduced to cyclopentanol by catalytic hydrogenation or by reaction with a suitable reductant, e.g. sodium borohydride. The 1,3-butadiene and the carbon monoxide used in this process may be obtained from the pyrolysis oil or the syngas according to the invention. Cylcopentanol is useful for the production of cyclopentyl acrylate and cyclopentyl methacrylate either by a usually acid-catalyzed esterification of acrylic or methacrylic acid, or by transesterification with methyl acrylate or methyl methacrylate, respectively.

### Cyclohexanol

Cyclohexanol can be produced together with cyclohexanone by the catalytic oxidation of cyclohexane with atmospheric oxygen in the presence of, e.g., a cobalt catalyst. This radical reaction proceeds via the unstable intermediate cyclohexane hydroperoxide, which decomposes into a mixture of the two products mentioned. From this mixture cyclohexanol can be separated by distillation. Cyclohexane used as starting material for the above oxidation, is commonly produced on a large scale by catalytic hydrogenation of benzene over a Raney nickel catalyst. Benzene may in turn be obtained from the pyrolysis oil according to the invention.

Alternatively, cyclohexanol can be produced by hydrogenation of phenol in the presence of a nickel catalyst. Phenol in turn is accessible by the so-called cumene process, which is an industrial procedure for the simultaneous production of phenol and acetone. The process involves converting benzene and propene via Friedel-Crafts alkylation to cumene, which is then oxidized by atmospheric oxygen to cumene hydroperoxide. Upon acidic work-up the hydroperoxide decomposes under rearrangement to phenol and acetone. Benzene used as a starting material may be obtained from the pyrolysis oil according to the invention.

Cylcohexanol is useful for the production of cyclohexyl acrylate and cyclohexyl methacrylate either by a usually acid-catalyzed esterification of acrylic or methacrylic acid, or by transesterification with methyl acrylate or methyl methacrylate, respectively.

### Higher oxo Alcohols, Such as Isononanol and 2-propyl-1-heptanol

Higher oxo alcohols are generally produced in an analogous manner as described above for shorter chain oxo alcohols (see, e.g., the production processes of n-propanol and 2-ethylhexanol outlined before). Thus, the initial reaction is a hydroformylation of a Cₙ-alkene. The resulting Cₙ₊₁-alkanal is then either hydrogenated to the corresponding Cₙ₊₁-alkanol, or first subjected to a self-aldol condensation to give the respective C₂₍ₙ₊₁₎-alkenal, which is hydrogenated to the C₂₍ₙ₊₁₎-alkanol. In particular the C₁-C₄-alkene obtainable from the pyrolysis oil according to the invention may be used as starting products for this procedure.

Important higher oxo alcohols are in particular isononanol and 2-propyl-1-heptanol. isononanol is a mixture of branched primary C₉-alcohols (main component: 3,5,5-trimethyl-1-hexanol) that can be produced by first hydroformylating a mixture of branched octenes (main component: diisobutylene) and afterwards hydrogenating the resulting C₉-aldehyldes. The mixtures of branched octenes are accessible by dimerization of isobutene and 1-butene, which in turn may both be obtained from the pyrolysis oil according to the invention.

The production of 2-propyl-1-heptanol, on the other hand, starts from n-pentanal, which is formed by hydroformylation of n-butene. Self-aldol condensation of the n-pentanal followed by catalytic hydrogenation of the resulting 2-propylhept-2-enal yields the racemic 2-propyl-1-heptanol. The n-butene used in this procedure may also be obtained from the pyrolysis oil according to the invention.

Isononanol and 2-propyl-1-heptanol are useful for the production of the corresponding isononyl or 2-propyl-1-heptyl (meth)acrylates either by a usually acid-catalyzed esterification of acrylic or methacrylic acid, or by transesterification with methyl acrylate or methyl methacrylate, respectively.

### Alkyl Acrylates

As mentioned before, alkyl acrylates, such as e.g. ethyl acrylate, n-butyl acrylate, isobutyl acrylate, 2-ethylhexyl acrylate, can be produced by acid-catalyzed esterification of acrylic acid with ethanol, n-butanol, isobutanol and 2-ethylhexanol respectively, wherein acrylic acid and said alcohols are obtainable by the processes described herein before.

Alternatively, alkyl acrylates, such as those explicitly mentioned above, may be prepared from acetylene, carbon monoxide and the respective alkanol by a Reppe reaction. Acetylene and the alkanols ethanol, n-butanol, isobutanol and 2-ethylhexanol are obtainable form cracking of the pyrolysis oil according to the invention, while carbon monoxide may be obtained from the syngas according to the invention.

### Tert-Butyl Acrylate

Tert-butyl acrylate is produced on a large scale by acid-catalyzed reaction of acrylic acid with isobutene at relatively low pressures, typically not exceeding 10 bar. Acrylic acid in turn is accessible by the process described above, while isobutene may be obtained from the pyrolysis oil according to the invention.

### Vinyl Acetate

The major industrial route to produce vinyl acetate involves the selective gas-phase oxidation of acetic acid with ethylene in the presence of oxygen. The conversion takes place in steam-heated tubular reactors typically at temperatures of about 150 to 160 °C and pressures of about 8-11 bar. Bimetallic palladium-gold shell catalysts are usually used as catalysts. The acetic acid used in this process can be produced by carbonylation of methanol based on the reaction of methanol with carbon monoxide in the presence of a catalyst, such as a rhodium or iridium carbonyl complex. The starting materials ethylene and carbon monoxide, on the other hand, may be obtained from the pyrolysis oil or the syngas according to the invention, while methanol is available from catalytic hydrogenation of syngas as described above.

### Vinyl Propionate

The major industrial route to produce vinyl propionate involves the reaction of acetylene with propionic acid in the presence of suitable catalysts, such as carbon and zinc salts. Propionic acid can be prepared by gas-phase oxidation of propanal in the presence of suitable catalysts, such as manganese salts or cobalt salts, such as manganese (II) propionate. Propanal can be produced by hydroformylation of ethylene. Alternatively, propionic acid can be prepared by hydrocarboxylation of ethylene with carbon monoxide and water in the presence of a nickel catalyst, such as nickel tetracarbonyl. The starting materials ethylene and carbon monoxide, on the other hand, may be obtained from the pyrolysis oil or the syngas according to the invention, while acetylene can be obtained from the gaseous fraction of the pyrolysis of plastic waste.

### Acrylonitrile

On an industrial scale acrylonitrile is produced by catalytic ammoxidation of propene, also known as the SOHIO process, which involves the conversion of propene with ammonia and oxygen. The propene used in this process may be obtained from the pyrolysis oil according to the invention.

### Acrylamide

The large-scale production of acrylamide is carried out by hydrolysis of acrylonitrile. The reaction can be catalyzed by a strong aqueous acid, usually sulfuric acid, by metal salts or by the enzyme nitrile hydratase. The starting compound acrylonitrile may be obtained from the SOHIO process described above.

In order to achieve the particular benefits of the invention, the relative amount of monomers M′, which are produced from a pyrolysis oil as a raw material, which is obtained by pyrolysis of organic plastic waste materials, is at least 20% by weight, in particular at least 30% by weight and especially at least 50% by weight, based on the total weight of monomers M subjected to a radical aqueous emulsion polymerization. The amount of monomers M′ may also be 100%, based on the total amount of monomers M to be polymerized. Typically, a mixture of monomers M′, which are produced from a pyrolysis oil as a raw material, which is obtained by pyrolysis of organic plastic waste materials, and monomers M″ which are produced from other feedstocks will be used in the radical aqueous emulsion polymerization.

In the radical aqueous emulsion polymerization, the monomers M to be polymerized typically comprise at least 80% by weight, e.g. 80 to 100% by weight, or 80 to 99.9% by weight, in particular 85 to 99.9% by weight, based on the total weight of the monomers M, of one or more ethylenically unsaturated monomers M, which have a limited solubility in water, in particular a solubility in deionized water, which does not exceed 50 g/L at 20 °C and 1 bar. These monomers are hereinafter referred to as monomers M1.

Examples of such monomers M1 include in particular
acrylate esters and methacrylate esters, in particular esters of acrylic and esters of methacrylic acid with alkanols having 1 to 18 C atoms, such as methyl acrylate, ethyl acrylate, n-propyl acrylate, isopropyl acrylate, n-butyl acrylate, 2-butyl acrylate, tert-butyl acrylate, n-pentyl acrylate, isopentyl acrylate, n-hexyl acrylate, n-heptyl acrylate, n-octyl acrylate, 2-ethylhexyl acrylate, 2-propylpentyl acrylate, n-decyl acrylate, 2-propylheptyl acrylate, C₁₀ isoamyl guerbet acrylate, 1-propylheptyl acrylate, lauryl acrylate and stearyl acrylate. Examples of C₁-C₂₀ alkyl esters of methacrylic acid include, but are not limited to methyl methacrylate, ethyl methacrylate, n-propyl methacrylate, isopropyl methacrylate, n-butyl methacrylate, 2-butyl methacrylate, tert-butyl methacrylate, n-pentyl methacrylate, isopentyl methacrylate, n-hexyl methacrylate, n-heptyl methacrylate, n-octyl methacrylate, 2-ethylhexyl methacrylate, 2-propylpentyl methacrylate, n-decyl methacrylate, 2-propylheptyl methacrylate, C₁₀ isoamyl guerbet methacrylate, 1-propylheptyl methacrylate, lauryl methacrylate and stearyl methacrylate;

esters of acrylic and/or methacrylic acid with cycloalkanols having 3 to 18 C atoms, in particular 5 to 10 C atoms, such as cyclopropylacrylate, cyclopentyl acrylate, cyclohexyl acrylate, 4-methylcyclohexyl acrylate, 4-tert-butylcyclohexyl acrylate, cyclopentyl methacrylate, cyclohexyl methacrylate, 4-methylcyclohexyl methacrylate and 4-tert-butylcyclohexyl methacrylate;
monovinylaromatic hydrocarbons such as styrene, 2-methylstyrene, 4-methylstyrene, 2-n-butylstyrene, 4-n-butylstyrene or 4-n-decylstyrene;
conjugated alkadienes, such as butadiene or isoprene;
olefins and haloolefins such as ethylene, propene, vinyl chloride, and vinylidene chloride;
vinyl esters and allyl esters of saturated C₁-C₁₂ alkanoic acids such as vinyl formate, vinyl acetate, vinyl propionate, vinyl butyrate, vinyl hexanoate, vinyl octanoate, vinyl laurate, vinyl stearate, vinyl esters of Versatic acid, allyl formate, allyl acetate, allyl propionate, allyl butyrate or allyl laurate.

Preferred monomers M1 are esters of acrylic with alkanols having 1 to 10 C atoms esters of methacrylic acid with alkanols having 1 to 10 C atoms, monovinylaromatic hydrocarbon compounds, specifically styrene, conjugated alkadienes, specifically butadiene, vinyl esters of saturated C₁-C₁₂ alkanoic acids, specifically vinylacetate, vinyl propionate and vinyl versatete, and olefins, specifically ethylene and combinations thereof.

In particular, the monomers M1 are one of the following monomer combinations (1) to (5):
(1) at least one monovinyl aromatic monomer, at least one acrylate ester and optionally one or more methacrylate esters,
(2) at least one acrylate ester and at least one methacrylate ester,
(3) at least one monovinyl aromatic monomer, butadiene and optionally one or more monomers selected from acrylate esters and methacrylate esters
(4) at least one vinylester of a C₂-C₁₂-alkanoic acid;
(5) at least one vinylester of a C₂-C₁₂-alkanoic acid and at least one C₂-C₄-monoolefine and optionally one of acrylate esters and methacrylate esters.

Besides the monomers M1, the monomers to be polymerized in the aqueous radical emulsion polymerization may comprise one or more ethylenically unsaturated monomers which are different from the monomers M2, which are in particular selected from the monomers M2, M3 and M4 described hereinafter. The total amount of these monomers is typically in the range of 0 to 20% by weight, e.g. in the range of 0.1 to 20% by weight, based on the total weight of the monomers M.

As explained above, the monomers M comprise the monomers M′ preferably in an amount of at least 10% by weight, in particular at least 20% by weight, more preferably at least 30% by weight, especially at least 50% by weight, or up to 100% by weight, based on the total weight of monomers M. Typically the monomers M′ are chosen from the aforementioned monomers M1 but they may also be chosen from the monomers M2. In particular, the monomers M′ are used in such an amount, that the relative amount of carbon atoms stemming from the pyrolysis oil in the monomers M is at least 20 mol %, in particular at least 50 mol-%, based on the total amount of carbon atoms in the monomers M.

Examples for monomers M.2 are monoethylenically unsaturated acidic monomers M2.a such as
monoethylenically unsaturated monocarboxylic acids having 3 to 8 C atoms such as acrylic acid, methacrylic acid or itaconic acid;
ethylenically unsaturated sulfonic acids and their salts such as vinylsulfonic acid, allylsulfonic acid, sulfoethyl acrylate, sulfoethyl methacrylate, sulfopropyl acrylate, sulfopropyl methacrylate, 2-hydroxy-3-acryloyloxypropylsulfonic acid, 2-hydroxy-3-methacryloyloxypropylsulfonic acid, styrenesulfonic acids, and 2-acrylamido-2-methylpropanesulfonic acid, especially their salts, more particularly their sodium salts and their ammonium salts;
ethylenically unsaturated phosphonic acid and ethyleneically unsaturated phosphoric acids and their salts such as vinylphosphonic acid, allylphosphonic acid, phosphoethyl acrylate, phosphoethyl methacrylate, phosphopropyl acrylate, phosphopropyl methacrylate, phospho-oligo(C₂-C₃-alkyleneether)acrylate, phospho-oligo(C₂-C₃-alkyleneether)methacrylate, especially their salts, more particularly their sodium salts and their ammonium salts;

and monoethylenically unsaturated neutral monomers M2.b, such as
primary amides of monoethylenically unsaturated monocarboxylic acids having 3 to 8 C atoms such as acrylamide and methacrylamide;
monoethylenically unsaturated monomers which carry urea groups or keto groups, such as 2-(2-oxoimidazolidin-1-yl)ethyl (meth)acrylate, 2-ureido(meth)acrylate, N-[2-(2-oxo-oxazolidin-3-yl)ethyl] methacrylate, acetoacetoxyethyl acrylate, acetoacetoxypropyl methacrylate, acetoacetoxybutyl methacrylate, 2-(acetoacetoxy)ethyl methacrylate, diacetoneacrylamide (DAAM) and diacetonemethacrylamide;
esters of acrylic and/or methacrylic acid with alkandiols having 2 to 4 C atoms, such as 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate or 4-hydroxybutyl methacrylate;

Preferably, the monomers M contain not more than 5 pphm, e.g. 0.1 to 5 pphm of monoethylenically unsaturated monomers M2.a having an acid group. Preferably, the monomers M contain not more than 10 pphm of monomers M2b, e.g. 0 to 10 pphm or 0.1 to 10 pphm of monomers M2.b.

In addition to the aforementioned monoethylenically unsaturated monomers monomers M1 and M2, the monomers M may comprise a small amount of ethylenically unsaturated monomers M3, which bear at least 2, e.g. 2 to 6 non-conjugated ethylenically unsaturated double bonds. These monomers will result in a crosslinking of the polymer chain during polymerization and thus are referred to as crosslinking monomers M3. Exemplary crosslinking monomers include divinylbenzene, diesters or triesters of dihydric and trihydric alcohols with monoethylenically unsaturated C₃-C₆ monocarboxylic acids, e.g., di(meth)acrylates, tri(meth)acrylates), and tetra(meth)acrylates, e.g. alkylene glycol diacrylates and dimethacrylates, such as ethylene glycol diacrylate, 1,3-butylene glycol diacrylate, 1,4-butylene glycol diacrylate and propylene glycol diacrylate, trimethylolpropan triacrylate and trimethacrylate, pentaerythrit triacrylate and pentaerythrit tetraacrylate, but also vinyl and allyl esters of ethylenically unsaturated acids such as vinyl methacrylate, vinyl acrylate, allyl methacrylate, allyl acrylate, and divinyl and diallyl esters of dicarboxyilic acids, such as diallyl maleate and diallyl fumarate and also methylenebisacrylamide. The amount of said monomers M3 will usually not exceed 3 pphm and, if present, is in particular in the range of 0.01 to 3 pphm.

In addition to the aforementioned monoethylenically unsaturated monomers monomers M1 and M2, the monomers M may comprise a small amount of ethylenically unsaturated monomers M4, which have one unsaturated double bond and a further reactive group susceptible to a post-crosslinking reaction, including
monoethylenically unsaturated monomers containing a keto group, e.g., aceto-acetoxyethyl(meth)acrylate or diacetonacrylamide;
monoethylenically unsaturated monomers, which bear an epoxy group, such as monoeglycidyl allyl ether, glycidyl acrylate, glycidyl methacrylate, 2-glycidyloxyethyl acrylate, 2-glycidyloxyethyl methacrylate, 3-glycidyloxypropyl acrylate, 3-glycidyloxypropyl methacrylate, 4-glycidyloxybutyl acrylate 4-glycidyloxybutyl methacrylate, 3,4-epoxybutyl acrylate, 3,4-epoxybutyl methacrylate, 4,5-epoxypent-2-yl acrylate or 4,5-epoxypent-2-yl methacrylate with preference given to epoxy functionalized (meth)acrylate monomers;
N-alkylolamides of α,β-monoethylenically unsaturated carboxylic acids having 3 to 10 carbon atoms and esters thereof with alcohols having 1 to 4 carbon atoms, e.g. N-methylol acrylamide and N-methylol methacrylamide;
unsaturated silan functional monomers, e.g. monomers which in addition to an ethylenically unsaturated double bond bear at least one mono-, di- and/or tri-C₁-₄-alkoxysilane group, such as vinyl trimethoxysilane, vinyl triethoxysilane, methacryloxyethyl trimethoxysilane, methacryloxyethyl triethoxysilane, and mixtures thereof.

The amount of said monomers M4 will usually not exceed 10 pphm and is in particular in the range of 0.01 to 10 pphm.

According to the invention the monomers M are polymerized in a radical emulsion aqueous emulsion polymerization, in particular in a free radical emulsion polymerization. This technique has been exhaustively described in the art, and is therefore well known to the skilled person [cf., e.g., Encyclopedia of Polymer Science and Engineering, vol. 8, pages 659 to 677, John Wiley & Sons, Inc., 1987; D. C. Blackley, Emulsion Polymerisation, pages 155 to 465, Applied Science Publishers, Ltd., Essex, 1975; D. C. Blackley, Polymer Latices, 2nd edition, vol. 1, pages 33 to 415, Chapman & Hall, 1997; H. Warson, The Applications of Synthetic Resin Emulsions, pages 49 to 244, Ernest Benn, Ltd., London, 1972; J. Piirma, Emulsion Polymerisation, pages 1 to 287, Academic Press, 1982; F. Hölscher, Dispersionen synthetischer Hochpolymerer, pages 1 to 160, Springer-Verlag, Berlin, 1969, and patent specification DE-A 40 03 422].

The radically initiated aqueous emulsion polymerization is normally accomplished by dispersing the ethylenically unsaturated monomers in aqueous medium, generally with accompanying use of surfactants, such as emulsifiers and/or protective colloids, and polymerizing them by means of at least one polymerization initiator, in particular a water-soluble radical polymerization initiator. These surfactants typically comprise emulsifiers and provide micelles in which the polymerization occurs, and which serve to stabilize the monomer droplets during aqueous emulsion polymerization and also growing polymer particles. The surfactants used in the emulsion polymerization are usually not separated from the polymer dispersion, but remain in the aqueous polymer dispersion obtainable by the emulsion polymerization of the monomers M.

The free-radically initiated aqueous emulsion polymerization is triggered by means of a free-radical polymerization initiator (free-radical initiator). These may in principle be peroxides or azo compounds. Of course, redox initiator systems are also useful. Peroxides used may, in principle, be inorganic peroxides, such as hydrogen peroxide or peroxodisulfates, such as the mono- or di-alkali metal or ammonium salts of peroxodisulfuric acid, for example the mono- and disodium,-potassium or ammonium salts, or organic peroxides such as alkyl hydroperoxides, for example tert-butyl hydroperoxide, p-menthyl hydroperoxide or cumyl hydroperoxide, and also dialkyl or diaryl peroxides, such as di-tert-butyl or di-cumyl peroxide. Azo compounds used are essentially 2,2′-az-obis(isobutyronitrile), 2,2′-azobis(2,4-dimethylvaleronitrile) and 2,2′-azobis(amidinopropyl) dihydrochloride (AIBA, corresponds to V-50 from Wako Chemicals). Suitable oxidizing agents for redox initiator systems are essentially the peroxides specified above. Corresponding reducing agents which may be used are sulfur compounds with a low oxidation state, such as alkali metal sulfites, for example potassium and/or sodium sulfite, alkali metal hydrogensulfites, for example potassium and/or sodium hydrogensulfite, alkali metal metabisulfites, for example potassium and/or sodium metabisulfite, formaldehydesulfoxylates, for example potassium and/or sodium formaldehydesulfoxylate, alkali metal salts, specifically potassium and/or sodium salts of aliphatic sulfinic acids and alkali metal hydrogensulfides, for example potassium and/or sodium hydrogensulfide, salts of polyvalent metals, such as iron(II) sulfate, iron(II) ammonium sulfate, iron(II) phosphate, ene diols, such as dihydroxymaleic acid, benzoin and/or ascorbic acid, and reducing saccharides, such as sorbose, glucose, fructose and/or dihydroxyacetone. Preferred free-radical initiators are inorganic peroxides, especially peroxodisulfates, and redox initiator systems. In general, the amount of the free-radical initiator used, based on the total amount of monomers M, is 0.01 to 5 pphm, preferably 0.1 to 3 pphm.

The amount of free-radical initiator required in the process of the invention for the emulsion polymerization M can be initially charged in the polymerization vessel completely. However, it is also possible to charge none of or merely a portion of the free-radical initiator, for example not more than 30% by weight, especially not more than 20% by weight, based on the total amount of the free-radical initiator required in the aqueous polymerization medium and then, under polymerization conditions, during the free-radical emulsion polymerization of the monomers M to add the entire amount or any remaining residual amount, according to the consumption, batchwise in one or more portions or continuously with constant or varying flow rates.

Preferably, the radical emulsion polymerization of the monomers M is performed by a so-called feed process, which means that at least 90%, in particular at least 95% or the total amount of the monomers to be polymerized are metered to the polymerization reaction under polymerization conditions during a metering period P. The duration of the period P may depend on the production equipment, the reactivity of the monomers and the polymerization initiator and the feed rate of the monomers (starved conditions vs. flooded conditions) and may vary from e.g. 20 minutes to 12 h. Frequently, the duration of the period P will be in the range from 0.5 h to 5 h, especially from 1 h to 4 h.

The term “polymerization conditions” is generally understood to mean those temperatures and pressures under which the free-radically initiated aqueous emulsion polymerization proceeds at sufficient polymerization rate. They depend particularly on the free-radical initiator used. Advantageously, the type and amount of the free-radical initiator, polymerization temperature and polymerization pressure are selected such that a sufficient amount of initiating radicals is always present to initiate or to maintain the polymerization reaction.

It may be suitable to establish the polymerization conditions and to initially charge at least a portion of the free-radical initiator into the polymerization vessel before the metering of the monomers M is started.

In some cases it has been found advantageous to perform the free-radical emulsion polymerization in the presence of seed latex. A seed latex is a polymer latex which is present in the aqueous polymerization medium before the metering of the monomers M is started. The seed latex may help to better adjust the particle size of the final polymer latex obtained in the free-radical emulsion polymerization of the invention.

Principally every polymer latex may serve as seed latex. For the purpose of the invention, preference is given to seed latices, where the particle size of the polymer particles is comparatively small. In particular, the Z average particle diameter of the polymer particles of the seed latex, as determined by dynamic light scattering at 20 °C (see below) is preferably in the range from 10 to 80 nm, in particular from 10 to 50 nm. Preferably, the polymer particles of the seed latex is made of ethylenically unsaturated monomers, which comprise at least 95% by weight, based on the total weight of the monomers forming the seed latex, of one or more monomers M1 as defined above. In the polymer particles of the seed latex particular comprise at least 95% by weight, based on the total weight of the monomers forming the seed latex, of at least one monomer M1 or of a mixture of at least two monomers M1.

For this, the seed latex is usually charged into the polymerization vessel before the metering of the monomers M is started. In particular, the seed latex is charged into the polymerization vessel followed by establishing the polymerization conditions, e.g. by heating the mixture to polymerization temperature. It may be beneficial to charge at least a portion of the free-radical initiator into the polymerization vessel before the metering of the monomers M is started. However, it is also possible to meter the monomers and the free-radical polymerization initiator in parallel to the polymerization vessel.

The amount of seed latex, calculated as solids, may frequently be in the range from 0.01 to 10% by weight, in particular from 0.1 to 5% by weight, based on the total weight of the monomers M to be polymerized.

The radical aqueous emulsion polymerization of the invention can be conducted at temperatures in the range from 0 to 170 °C. Temperatures employed are generally in the range from 50 to 120 °C, frequently from 60 to 120 °C and often from 70 to 110 °C. The free-radical aqueous emulsion polymerization of the invention can be conducted at a pressure of less than, equal to or greater than 1 atm (atmospheric pressure), and so the polymerization temperature may exceed 100 °C and may be up to 170 °C. Polymerization of the monomers is normally performed at ambient pressure but it may also be performed under elevated pressure. In this case, the pressure may assume values of 1.2, 1.5, 2, 5, 10, 15 bar (absolute) or even higher values. If emulsion polymerizations are conducted under reduced pressure, pressures of 950 mbar, frequently of 900 mbar and often 850 mbar (absolute) are established. Advantageously, the free-radical aqueous emulsion polymerization of the invention is conducted at ambient pressure (about 1 atm) with exclusion of oxygen, for example under an inert gas atmosphere, for example under nitrogen or argon.

The polymerization of the monomers M can optionally be conducted in the presence of chain transfer agents. Chain transfer agents are understood to mean compounds that transfer free radicals and which reduce the molecular weight of the or control chain growth in the polymerization. Examples of chain transfer agents are aliphatic and/or araliphatic halogen compounds, for example n-butyl chloride, n-butyl bromide, n-butyl iodide, methylene chloride, ethylene dichloride, chloroform, bromoform, bromotrichloromethane, dibromodichloromethane, carbon tetrachloride, carbon tetrabromide, benzyl chloride, benzyl bromide, organic thio compounds, such as primary, secondary or tertiary aliphatic thiols, for example ethanethiol, n-propanethiol, 2-propanethiol, n-butanethiol, 2-butanethiol, 2-methyl-2-propanethiol, n-pentanethiol, 2-pentanethiol, 3-pentanethiol, 2-methyl-2-butanethiol, 3-methyl-2-butanethiol, n-hexanethiol, 2-hexanethiol, 3-hexanethiol, 2-methyl-2-pentanethiol, 3-methyl-2-pentanethiol, 4-methyl-2-pentanethiol, 2-methyl-3-pentanethiol, 3-methyl-3-pentanethiol, 2-ethylbutanethiol, 2-ethyl-2-butanethiol, n-heptanethiol and the isomeric compounds thereof, n-octanethiol and the isomeric compounds thereof, n-nonanethiol and the isomeric compounds thereof, n-decanethiol and the isomeric compounds thereof, n-undecanethiol and the isomeric compounds thereof, n-dodecanethiol and the isomeric compounds thereof, n-tridecanethiol and isomeric compounds thereof, substituted thiols, for example 2-hydroxyethanethiol, aromatic thiols such as benzenethiol, ortho-, meta- or para-methylbenzenethiol, alkylesters of mercaptoacetic acid (thioglycolic acid), such as 2-ethylhexyl thioglycolate, alkylesters of mercaptopropionic acid, such as octyl mercapto propionate, and also further sulfur compounds described in Polymer Handbook, 3rd edition, 1989, J. Brandrup and E. H. Immergut, John Wiley & Sons, section II, pages 133 to 141, but also aliphatic and/or aromatic aldehydes, such as acetaldehyde, propionaldehyde and/or benzaldehyde, unsaturated fatty acids, such as oleic acid, dienes having nonconjugated double bonds, such as divinylmethane or vinylcyclohexane, or hydrocarbons having readily abstractable hydrogen atoms, for example toluene. Alternatively, it is possible to use mixtures of the aforementioned chain transfer agents that do not disrupt one another. The total amount of chain transfer agents optionally used in the process of the invention, based on the total amount of monomers M, will generally not exceed 1% by weight. However, it is possible, that during a certain period of the polymerization reaction the amount of chain transfer agent added to the polymerization reaction may exceed the value of 1% by weight, based on the total amount of monomers already added to the polymerization reaction.

The radical emulsion polymerization of the invention is usually effected in an aqueous polymerization medium, which, as well as water, comprises at least one surface-active substance (surfactant) for stabilizing the emulsion of the monomers and the polymer particles of the polymer latex.

The surfactant may be selected from emulsifiers and protective colloids. Protective colloids, as opposed to emulsifiers, are understood to mean polymeric compounds having molecular weights above 2000 Daltons, whereas emulsifiers typically have lower molecular weights. The surfactants may be anionic or nonionic or mixtures of non-ionic and anionic surfactants.

Anionic surfactants usually bear at least one anionic group, which is selected from phosphate, phosphonate, sulfate, and sulfonate groups. The anionic surfactants, which bear at least one anionic group, are typically used in the form of their alkali metal salts, especially of their sodium salts or in the form of their ammonium salts.

Preferred anionic surfactants are anionic emulsifiers, in particular those, which bear at least one sulfate or sulfonate group. Likewise, anionic emulsifiers, which bear at least one phosphate or phosphonate group may be used, either as sole anionic emulsifiers or in combination with one or more anionic emulsifiers, which bear at least one sulfate or sulfonate group.

Examples of anionic emulsifiers, which bear at least one sulfate or sulfonate group, are, for example,
the salts, especially the alkali metal and ammonium salts, of alkyl sulfates, especially of C₈-C₂₂-alkyl sulfates,
the salts, especially the alkali metal and ammonium salts, of sulfuric monoesters of ethoxylated alkanols, especially of sulfuric monoesters of ethoxylated C₈-C₂₂-alkanols, preferably having an ethoxylation level (EO level) in the range from 2 to 40,
the salts, especially the alkali metal and ammonium salts, of sulfuric monoesters of ethoxylated alkylphenols, especially of sulfuric monoesters of ethoxylated C₄-C₁₈-alkylphenols (EO level preferably 3 to 40),
the salts, especially the alkali metal and ammonium salts, of alkylsulfonic acids, especially of C₈-C₂₂-alkylsulfonic acids,
the salts, especially the alkali metal and ammonium salts, of dialkyl esters, especially di-C₄-C₁₈-alkyl esters of sulfosuccinic acid,
the salts, especially the alkali metal and ammonium salts, of alkylbenzenesulfonic acids, especially of C₄-C₂₂-alkylbenzenesulfonic acids, and
the salts, especially the alkali metal and ammonium salts, of mono- or disulfonated, alkyl-substituted diphenyl ethers, for example of bis(phenylsulfonic acid) ethers bearing a C₄-C₂₄-alkyl group on one or both aromatic rings. The latter are common knowledge, for example from U.S. Pat. No. 4,269,749, and are commercially available, for example as Dowfax® 2A1 (Dow Chemical Company).

Also suitable are mixtures of the aforementioned salts.

Preferred anionic surfactants are anionic emulsifiers, which are selected from the following groups:
the salts, especially the alkali metal and ammonium salts, of alkyl sulfates, especially of C₈-C₂₂-alkyl sulfates,
the salts, especially the alkali metal salts, of sulfuric monoesters of ethoxylated alkanols, especially of sulfuric monoesters of ethoxylated C₈-C₂₂-alkanols, preferably having an ethoxylation level (EO level) in the range from 2 to 40,
of sulfuric monoesters of ethoxylated alkylphenols, especially of sulfuric monoesters of ethoxylated C₄-C₁₈-alkylphenols (EO level preferably 3 to 40),
of alkylbenzenesulfonic acids, especially of C₄-C₂₂-alkylbenzenesulfonic acids, and
of mono- or disulfonated, alkyl-substituted diphenyl ethers, for example of bis(phenylsulfonic acid) ethers bearing a C₄-C₂₄-alkyl group on one or both aromatic rings.

Examples of anionic emulsifiers, which bear a phosphate or phosphonate group, include, but are not limited to the following, salts selected from the following groups:
the salts, especially the alkali metal and ammonium salts, of mono- and dialkyl phosphates, especially C₈-C₂₂-alkyl phosphates,
the salts, especially the alkali metal and ammonium salts, of phosphoric monoesters of C₂-C₃-alkoxylated alkanols, preferably having an alkoxylation level in the range from 2 to 40, especially in the range from 3 to 30, for example phosphoric monoesters of ethoxylated C₈-C₂₂-alkanols, preferably having an ethoxylation level (EO level) in the range from 2 to 40, phosphoric monoesters of propoxylated C₈-C₂₂-alkanols, preferably having a propoxylation level (PO level) in the range from 2 to 40, and phosphoric monoesters of ethoxylated-co-propoxylated C₈-C₂₂-alkanols, preferably having an ethoxylation level (EO level) in the range from 1 to 20 and a propoxylation level of 1 to 20,
the salts, especially the alkali metal and ammonium salts, of phosphoric monoesters of ethoxylated alkylphenols, especially phosphoric monoesters of ethoxylated C₄-C₁₈-alkylphenols (EO level preferably 3 to 40),
the salts, especially the alkali metal and ammonium salts, of alkylphosphonic acids, especially C₈-C₂₂-alkylphosphonic acids and
the salts, especially the alkali metal and ammonium salts, of alkylbenzenephosphonic acids, especially C₄-C₂₂-alkylbenzenephosphonic acids.

Further suitable anionic surfactants can be found in Houben-Weyl, Methoden der organischen Chemie [Methods of Organic Chemistry], volume XIV/1, Makromolekulare Stoffe [Macromolecular Substances], Georg-Thieme-Verlag, Stuttgart, 1961, p. 192-208.

Preferably, the surfactant comprises at least one anionic emulsifier, which bears at least one sulfate or sulfonate group. The at least one anionic emulsifier, which bears at least one sulfate or sulfonate group, may be the sole type of anionic emulsifiers. However, mixtures of at least one anionic emulsifier, which bears at least one sulfate or sulfonate group, and at least one anionic emulsifier, which bears at least one phosphate or phosphonate group, may also be used. In such mixtures, the amount of the at least one anionic emulsifier, which bears at least one sulfate or sulfonate group, is preferably at least 50% by weight, based on the total weight of anionic surfactants used in the process of the present invention. In particular, the amount of anionic emulsifiers, which bear at least one phosphate or phosphonate group does not exceed 20% by weight, based on the total weight of anionic surfactants used in the process of the present invention.

As well as the aforementioned anionic surfactants, the surfactant may also comprise one or more nonionic surface-active substances, which are especially selected from nonionic emulsifiers. Suitable nonionic emulsifiers are e.g. araliphatic or aliphatic nonionic emulsifiers, for example ethoxylated mono-, di- and trialkylphenols (EO level: 3 to 50, alkyl radical: C₄-C₁₀), ethoxylates of long-chain alcohols (EO level: 3 to 100, alkyl radical: C₈-C₃₆), and polyethylene oxide/polypropylene oxide homo- and copolymers. These may comprise the alkylene oxide units copolymerized in random distribution or in the form of blocks. Very suitable examples are the EO/PO block copolymers. Preference is given to ethoxylates of long-chain alkanols, in particular to those where the alkyl radical C₈-C₃₀ having a mean ethoxylation level of 5 to 100 and, among these, particular preference to those having a linear C₁₂-C₂₀ alkyl radical and a mean ethoxylation level of 10 to 50, and also to ethoxylated monoalkylphenols.

Preferably, the surfactant will be used in such an amount that the amount of surfactant is in the range from 0.2 to 5% by weight, especially in the range from 0.5 to 3% by weight, based on the monomers M to be polymerized.

The aqueous reaction medium in polymerization may in principle also comprise minor amounts (usually at most 5% by weight) of water-soluble organic solvents, for example methanol, ethanol, isopropanol, butanols, pentanols, but also acetone, etc. Preferably, however, the process of the invention is conducted in the absence of such solvents.

It is frequently advantageous when the aqueous polymer dispersion obtained on completion of polymerization of the monomers M is subjected to an after-treatment to reduce the residual monomer content. This after-treatment is effected either chemically, for example by completing the polymerization reaction using a more effective free-radical initiator system (known as postpolymerization), and/or physically, for example by stripping the aqueous polymer dispersion with steam or inert gas. Corresponding chemical and physical methods are familiar to those skilled in the art—see, for example, EP-A 771328, DE-A 19624299, DE-A 19621027, DE-A 19741184, DE-A 19741187, DE-A 19805122, DE-A 19828183, DE-A 19839199, DE-A 19840586 and DE-A 19847115. The combination of chemical and physical aftertreatment has the advantage that it removes not only the unconverted ethylenically unsaturated monomers but also other disruptive volatile organic constituents (VOCs) from the aqueous polymer dispersion.

In a preferred group of embodiments, the aqueous emulsion polymerization is carried out by the procedure described in EP 917 545.

In a preferred group of embodiments, the aqueous emulsion polymerization is carried out by the procedure described in EP 3157992.

In yet a preferred group of embodiments, the aqueous emulsion polymerization is carried out by the procedure described in EP 3523382.

In yet a preferred group of embodiments, the aqueous emulsion polymerization is carried out by the procedure described in WO 2020/002102.

In yet a preferred group of embodiments, the aqueous emulsion polymerization is carried out by the procedure described in WO 2001/014426.

In yet a preferred group of embodiments, the aqueous emulsion polymerization is carried out by the procedure described in WO 2020/249406.

The radical aqueous emulsion polymerization may be carried out by a single stage or by a multistage emulsion polymerization, in particular an aqueous radical emulsion polymerization, of a monomer composition M. The term “multistage” in the context of aqueous emulsion polymerization is well understood to mean that the relative concentration of the monomers in the monomer composition M added to the polymerization reaction is altered at least once during the aqueous emulsion polymerization. Such a procedure results in at least two polymer populations of different monomer compositions in the polymer particles of the latex. For example, it will be possible to change the monomer composition such that the multistage latex polymer features populations having different glass transition temperatures or a glass transition temperature (T_{g}) gradient. It may also be possible to change the monomer composition such that the multistage latex polymer features populations having different concentrations of polymerized acidic monomers, such as monomers M2.a or a concentration gradient of monomers M2.a.

During the addition of the monomers M, the type of monomers and/or the relative amounts thereof can be altered continuously or stepwise. However, it is also possible that the type and relative amounts of monomers M, which are added to the polymerization reaction remains constant. For example, it is possible that the ratio of monomers M1 and M2 increases or decreases during the addition.

Preferably, the aqueous polymer latex of the carboxylated polymer is prepared by a radical aqueous emulsion polymerization by the so-called feed method, where during the feeding of the monomer composition M, where at least 90% of the monomer composition M to be polymerised are metered to the polymerisation reaction under polymerisation conditions during a metering period P, and where the composition of the portion of the monomer composition M, which is metered to the polymerisation reaction under polymerisation conditions is changed at least once during the metering period P.

The concentration of the polymer contained in the aqueous polymer dispersion is frequently in the range from 20 to 70% by weight, in particular in the range from 30 to 65% by weight, especially in the range from 40 to 65% by weight, based in on the total weight of the aqueous polymer dispersion.

In the aqueous polymer dispersion obtained by the process of the invention, the dispersed polymers are in the form of polymer particles. The polymer particles typically have an average diameter in the range from 30 to 1000 nm, in particular in the range from 40 to 900 nm and especially in the range from 50 to 800 nm. The average particle diameter as referred herein relates to the Z average particle diameter as determined by means of photon correlation spectroscopy (PCS), also known as quasielastic light scattering (QELS) or dynamic light scattering (DLS). The measurement method is described in the ISO 13321: 1996 standard. The determination can be carried out using an HPPS (High Performance Particle Sizer). For this purpose, a sample of the aqueous polymer latex will be diluted and the dilution will be analysed. In the context of DLS, the aqueous dilution may have a polymer concentration in the range from 0.001 to 0.5% by weight, depending on the particle size. For most purposes, a proper concentration will be 0.01% by weight. However, higher or lower concentrations may be used to achieve an optimum signal/noise ratio. The dilution can be achieved by addition of the polymer latex to water or an aqueous solution of a surfactant in order to avoid flocculation. Usually, dilution is performed by using a 0.1% by weight aqueous solution of a non-ionic emulsifier, e.g. an ethoxylated C16/C18 alkanol (degree of ethoxylation of 18), as a diluent. Measurement configuration: HPPS from Malvern, automated, with continuous-flow cuvette and Gilson autosampler. Parameters: measurement temperature 20.0 °C; measurement time 120 seconds (6 cycles each of 20 s); scattering angle 173°; wave-length laser 633 nm (HeNe); refractive index of medium 1.332 (aqueous); viscosity 0.9546 mPa·s. The measurement gives an average value of the second order cumulant analysis (mean of fits), i.e. Z average. The “mean of fits” is an average, intensity-weighted hydrodynamic particle diameter in nm.

The polymers in the polymer dispersion may have a monomodal particle size distributions, including narrow and broad monomodal particle size distributions but also multi-modal particle size distributions, depending on the desired purpose. The particle size distribution is characterized by the polydispersity index, which is a dimensionless number calculated from a simple 2 parameter fit to the correlation data of the cumulant analysis. The calculation is normally done as described in ISO 13321: 1996. Frequently, the PDI will be the range of 0.1 to 5.

Depending on the desired use, the polymer dispersions may have glass transition temperatures in a very broad range, e.g. in the range of −60 to 150 °C. They may have more than one phase, e.g. 2, 3 or 5 different phases having identical or different glass transition temperatures. The glass transition temperature can be determined by the DSC method (differential scanning calorimetry, 20 K/min, midpoint measurement) in accordance to DIN 53765: 1994-03 or ISO 11357-2, with sample preparation preferably to DIN EN ISO 16805: 2005.

The pH of the polymer dispersion may range from acidic to alkaline pH values, depending on the desired purpose and is frequently in the range of pH 2 to pH 10 or may be even higher e.g. up to pH 12.

The polymer dispersion obtained by the radical aqueous emulsion polymerization of the monomers M can be tailored to the desired purpose by choosing proper compositions of the monomers M. In particular, the polymer dispersions obtained by the radical aqueous emulsion polymerization of the monomers M can be used as binders in coating compositions, including masonry paints, interior paints, paints for wood coating and wood stains, and coating compositions for concrete and cement fiber board, as binder in paper coatings, as modifiers in hydraulically binding construction materials, such as concrete, plaster, and mortar, as binders in waterproofing membranes, as binders in flexible roofing, as binders for fiber bonding, and in adhesives, including e.g. pressure sensitive adhesives, construction adhesives and laminating adhesives.

Particular examples of polymer dispersions which can be produced by using the monomers M are described in EP 917 545, EP 3157992, EP 3523382, WO 2020/002102, WO 2001/014426 and WO 2020/249406, in particular the production examples described therein, in particular if the examples are carried out in industrial scale.

Carrying out the emulsion polymerization described in example E2 of WO 01/14426 in production scale (e.g. a 50 m³ reactor) by using 2-ethylhexyl acrylate and methyl methacrylate obtained from pyrolysis oil feedstock (=approx. 95 wt. % of the polymer is based on recycled material) results in an aqueous polymer dispersion having similar properties as the polymer latex of example E2. Moreover, by using pyrolysis oil feedstock for the production of these monomers the number of batches between the necessary maintenance and cleaning cycles can be increased from an average of 20 to an average of 24, which is an increase of about than 20% on average compared to carrying out the process with these monomers from a classic fossil naphtha feedstock.

Carrying out the emulsion polymerization described in example 1 of WO 2020/249406 in production scale (e.g. a 60 m³ reactor) by using butadiene and styrene obtained from pyrolysis oil feedstock (=approx. 95 wt. % of the polymer is based on recycled material) results in an aqueous polymer dispersion having similar properties as the polymer latex of example 1. Moreover, by using pyrolysis oil feedstock for the production of these monomers the number of batches between the necessary maintenance and cleaning cycles can be increased from an average of 90 to an average of 105, which is an increase of about 15% compared to carrying out the process with these monomers from a classic fossil feedstock.

Carrying out the emulsion polymerization described in example 1 of EP 3157992 in production scale (e.g. a 50 m³ reactor) by using styrene and n-butyl acrylate obtained from pyrolysis oil feedstock (=approx. 83 wt. % of the polymer is based on recycled material) results in an aqueous polymer dispersion having similar properties as the polymer latex of example 1. Moreover, by using pyrolysis oil feedstock for the production of these monomers the number of batches between the necessary maintenance and cleaning cycles can be increased from an average of 26 to an average of 31, which is an increase of about 20% compared to carrying out the process with these monomers from a classic fossil feedstock.

Carrying out the emulsion polymerization described in example of EP 3523382 in production scale (e.g. a 25 m³ reactor) by using styrene obtained from pyrolysis oil feedstock (=approx. 78 wt. % of the polymer is based on recycled material) results in an aqueous polymer dispersion having similar properties as the polymer dispersion C1 of example 3. Moreover, by using pyrolysis oil feedstock for the production of styrene the number of batches between the necessary maintenance and cleaning cycles can be increased from an average of 8 to an average of 10, which is an increase of about 25% compared to carrying out the process with these monomers from a classic fossil feedstock.

1.-16. (canceled)

17. A process for producing an aqueous polymer dispersion by radical aqueous emulsion polymerization of at least one ethylenically unsaturated monomer M, where a portion of the monomers M to be polymerized are ethylenically unsaturated monomers M′ produced from a pyrolysis oil as a raw material, which is obtained by pyrolysis of plastic waste materials.

18. The process of claim 17, where the pyrolysis oil has at least one of the following properties:
(α) a content of sulfur, selected from H₂S, elemental sulfur and organic sulfur, at least 5 ppm, determined according to ASTM D 7183-18 and calculated as elemental sulfur;
(β) a content of organic nitrogen of at least 20 ppm, determined according to ASTM D 5762-18 and calculated as elemental nitrogen; or
(χ) a content of organic oxygen of at least 50 ppm, determined according to ASTM D 7423-17 and calculated as elemental oxygen.

19. The process of claim 17, where the pyrolysis oil is obtained from mixed waste plastic material.

20. The process of claim 19, where the pyrolysis oil is subjected to a steam cracking where C₂-C₄ olefin fractions, >C₄ olefin fractions and benzene or methyl benzene fractions are obtained, which are converted into the desired monomers M′.

21. The process of claim 17, where the pyrolysis oil is obtained from waste rubber, in particular from waste tire material.

22. The process of claim 21, where the pyrolysis oil is subjected to a gasification step where a syngas fraction is obtained comprising a mixture of carbon monoxide and hydrogen, which is used in the production of the ethylenically unsaturated monomers M′.

23. The process of claim 22, where the syngas fraction is converted into methanol, which is subsequently used in the production of the ethylenically unsaturated monomers M′.

24. The process of claim 20, which comprises the following steps:
i) providing a pyrolysis oil form organic waste materials selected from mixed waste plastic;
ii) providing a full range naphtha from the pyrolysis oil obtained in step i);
iii) subjecting the full range naphtha to a steam cracking where C₂-C₄ olefin fractions, >C₄ olefin fractions and benzene or methyl benzene fractions are obtained; and optionally
iv) converting at least one of the fractions obtained in step ii) into an ethylenically unsaturated monomer M′; or
ii.a) subjecting the pyrolysis oil to a gasification step to obtain a syngas comprising a mixture of carbon monoxide and hydrogen,
iii. a) using the syngas fraction obtained in step ii.a) in the production of an ethylenically unsaturated monomer M′; and
v) conducting a radical aqueous emulsion polymerization of at least one ethylenically unsaturated monomer M, where at least 20% by weight of the monomers M to be polymerized are selected from the group consisting of the monomers M′ obtained in step iv), or step iii.a), one or more of the C₂-C₄ olefins obtained in step iii) and combinations thereof.

25. The process of claim 20, where in the production of the ethylenically unsaturated monomer at least one of the following fractions or combinations of fractions (a)-(f) obtained from steam cracking or gasification are used:
(a) a C₂-C₄-olefin fraction and the benzene fraction or methylbenzene fraction;
(b) C₂-C₄-olefin fraction;
(c) the syngas fraction;
(d) a C₂-C₄-olefin fraction and the syngas fraction;
(e) a >C₄ olefin fraction;
(f) a C₂-C₄-olefin fraction, a >C₄ olefin fraction and the syngas fraction.

26. The process of claim 17, where the ethylenically unsaturated monomers M′ are selected from the group consisting of monovinyl aromatic monomers, divinyl aromatic monomers, butadiene, acrylate esters, methacrylate esters, acrylic acid, methacrylic acid, amides of acrylic acid, amides of methacrylic acid, acrylonitrile, vinyl esters of C₂-C₁₂-alkanoic acids, allyl esters of C₂-C₁₂-alkanoic acids and mixtures thereof.

27. The process of claim 17 where at least 20% by weight of the monomers M are selected from ethylenically unsaturated monomers M′ produced from a pyrolysis oil as a raw material, which is obtained by pyrolysis of organic plastic waste materials.

28. The process of claim 17, where the monomers M to be polymerized comprise at least 80% by weight, based on the total weight of the monomers M, one of the following monomer combinations (1) to (5):
(1) at least one monovinyl aromatic monomer, at least one acrylate ester and optionally one or more methacrylate esters,
(2) at least one acrylate ester and at least one methacrylate ester,
(3) at least one monovinyl aromatic monomer, butadiene and optionally one or more monomers selected from acrylate esters and methacrylate esters
(4) at least one vinylester of a C₂-C₁₂-alkanoic acid;
(5) at least one vinylester of a C₂-C₁₂-alkanoic acid and at least one C₂-C₄-olefin.

29. The process of claim 26, where the acrylate esters are selected from C₁-C₁₈ alkyl acrylates and C₅-C₁₈ cycloalkyl acrylates and where the methacrylate esters are selected from C₁-C₁₈ alkyl methacrylates and C₅-C₁₈ cycloalkyl methacrylates.

30. The process of claim 17, where the relative amount of carbon atoms stemming from the pyrolysis oil in the monomers M is at least 20 mol %.

31. The process of claim 17, where the radical aqueous emulsion polymerization of the ethylenically unsaturated monomers M is carried out in industrial scale.

32. The use of ethylenically unsaturated monomers M′ produced from a pyrolysis oil as a raw material, which is obtained by pyrolysis of plastic waste materials, which are in particular selected from mixed waste plastic and waste rubber, for increasing the time period between the purification cycles of reaction vessels used in the industrial scale radical aqueous emulsion polymerization of ethylenically unsaturated monomers.
